(19) United States
(12) Patent Application Publication
(10) Pub. No.: US 2014/0088176 A1
(43) Pub. Date: Mar. 27, 2014

(54) GENE TRANSFER FOR REGULATING SMOOTH MUSCLE TONE

(71) Applicants: George J. Christ, Lewisville, NC (US); Kelvin Davies, New York, NY (US); Arnold Melman, Ardsley, NY (US)

(72) Inventors: George J. Christ, Lewisville, NC (US); Kelvin Davies, New York, NY (US); Arnold Melman, Ardsley, NY (US)

(73) Assignee: Albert Einstein College of Medicine of Yeshiva University, Bronx, NY (US)

(21) Appl. No.: 13/845,324

(22) Filed: Mar. 18, 2013

Related U.S. Application Data
(63) Continuation of application No. 10/579,705, filed on Oct. 31, 2008, now abandoned.

(51) Int. Cl. A61K 38/17

(52) U.S. Cl. CPC A61K 38/1709
USPC 514/44 R

(57) ABSTRACT

The invention provides methods of regulating smooth muscle tone in a subject, comprising the introduction, into smooth muscle cells of the subject, of a DNA sequence encoding a potassium channel protein involved in the regulation of smooth muscle tone, and expression of the DNA sequence in a sufficient number of smooth muscle cells of the subject to regulate smooth muscle tone in the subject. The invention provides methods of gene transfer for treating erectile dysfunction, bladder dysfunction, and other smooth muscle disorders.

### BACKGROUND OF THE INVENTION

There are many physiological dysfunctions or disorders which are caused by the deregulation of smooth muscle tone. Included among these are asthma; benign hyperplasia of the prostate gland (BPH); coronary artery disease; erectile dysfunction; genitourinary dysfunctions of the bladder, endopelvic fascia, prostate gland, ureter, urethra, urinary tract, and vas deferens; irritable bowel syndrome; migraine headaches; premature labor; Raynaud's syndrome; varicose veins; and thromboangitis obliterans.

Among these dysfunctions, erectile dysfunction is a common illness that is estimated to affect 10 to 30 million men in the United States (Feldman, et al., Journal of Clinical Epidemiology, 47(5):457-67, 1994; and Anonymous, International Journal of Impotence Research, 5(4):181-234, 1993). Among the primary disease-related causes of erectile dysfunction are aging, atherosclerosis, chronic renal disease, diabetes, hypertension and antihypertensive medication, pelvic surgery and radiation therapy, and psychological anxiety (Feldman, et al., Journal of Clinical Epidemiology, 47(5):457-67, 1994). Direct cures for the vascular ravages of these manifold and multifaceted disease states are unlikely to occur in the near future.

The last decade has witnessed the development of several treatment modalities to directly restore diminished erectile capacity. However, most currently-available therapies are either nonspecific (e.g., hormonal therapy), of limited overall success (e.g., vacuum erection devices), invasive (e.g., intracorporal injection therapy), or non-reversible and expensive (e.g., penile prosthetic implant surgery). Despite these therapeutic limitations, the approval by the U.S. Food and Drug Administration (FDA) of CAVERJECT® (Jul. 6, 1995) for intracavernous treatment of erectile dysfunction, of MUSE® (Nov. 19, 1996) for intraurethral drug administration in the treatment of erectile dysfunction, and of VIAGRA® (Mar. 27, 1998) and LEVITRA® (Aug. 19, 2003) as oral therapeutic agents for treatment of erectile dysfunction, represent major steps forward. The magnitude of the problem of erectile dysfunction, and the desire for more effective therapies, are highlighted by the number of prescriptions written for VIAGRA®. In essence, these acts of the U.S. Federal Government have resulted in the formal recognition of the medical nature of the problem of erectile dysfunction, and, furthermore, have legitimized its clinical treatment.

Studies have documented that altered corporal smooth muscle tone, resulting in either heightened contractility or impaired relaxation, is a proximal cause of erectile dysfunction in a large proportion of impotent men. These studies have further indicated that complete relaxation of corporal smooth muscle is both a necessary and sufficient condition to restore erectile potency, unless severe arterial disease or congenital structural abnormalities exist, which occur in only a minority of patients. The efficacy of recently-approved therapies for treating erectile dysfunction, which involve agents for directly or indirectly bringing about smooth-muscle relaxation—including PGE₁ (CAVERJECT®, EDEX®, and MUSES), Sildenafil (VIAGRA®) and Vardenafil (LEVITRA®)—verifies the validity of this supposition.

The critical role in erectile function played by corporal smooth muscle cells makes them an excellent target for molecular intervention in the treatment of erectile dysfunction. Previous efforts have focused on techniques for gene transfer into vascular smooth muscle cells as a basis for the potential therapy of several cardiovascular diseases. Among these are atherosclerosis, vasculitis, restenosis after balloon angioplasty, and pulmonary hypertension. These initial studies have provided important information on the efficiency and persistence of gene-transfer methods in smooth muscle cells (Finkel, et al., FASEB Journal, 9:843-51, 1995; Pozeg, et al., Circulation 107: 2037-44, 2003). Because erectile dysfunction is largely caused by altered smooth muscle tone, a method of gene transfer which targets the genes involved in the alteration of smooth muscle tone is extremely desirable. A successful method of gene transfer for alleviating erectile dysfunction is in great demand, as it would be a preferred alternative to currently-used methods.

Abnormal bladder function is another common problem that significantly affects the quality of life of millions of men and women in the United States. Many common diseases (e.g., BPH, diabetes mellitus, multiple sclerosis, and stroke) alter normal bladder function. Significant untoward changes in bladder function are also a normal result of advancing age.

There are two principal clinical manifestations of altered bladder physiology: the atonic bladder and the overactive bladder (Abrams P, et al., Neurourol. Urodyn. 21(2): 167-78, 2002). The atonic bladder has diminished capacity to empty its urine contents because of ineffective contractility of the detrusor smooth muscle (the smooth muscle of the bladder wall). In the atonic state, diminished smooth muscle contractility is implicated in the etiology of bladder dysfunction. Thus, it is not surprising that pharmacological modulation of smooth muscle tone is insufficient to correct the underlying problem. In fact, the prevailing method for treating this condition uses clean intermittent catheterization; this is a successful means of preventing chronic urinary tract infection, pyelonephritis, and eventual renal failure. As such, treatment of the atonic bladder ameliorates the symptoms of disease, but does not correct the underlying cause.

Conversely, the overactive bladder contracts spontaneously; this may result in urge incontinence, where the individual is unable to control the passage of urine. The overactive bladder is a more difficult clinical problem to treat than the atonic bladder. Medications that have been used to treat this condition are usually only partially effective, and have significant side effects that limit the patient's use of and enthusiasm to continue with the drug. The currently-accepted treatment options (e.g., oxybutynin and tolteradine) are largely nonspecific, and most frequently involve blockade of the muscarinic-receptor pathways and/or the calcium channels on the bladder myocytes. Given the central importance of these two pathways in the cellular functioning of many organ systems in the body, such nonspecific therapeutic strategies are not only crude methods for modulating bladder smooth muscle tone; rather, because of their very mechanism(s) of action, they are also virtually guaranteed to have significant and undesirable systemic effects. Accordingly, there is a great need for improved treatment options for bladder dysfunction.

There are some physiologically-relevant parallels between penile physiology and bladder physiology which bear comparison. For example, the tone of the detrusor smooth muscle plays a role in the etiology of bladder dysfunction that is similar to the well-characterized role of corporal smooth muscle tone in erectile dysfunction. In particular, the overactive bladder is characterized by heightened contractility, while the atonic bladder is characterized by impaired contractility. Pharmacological therapy for treating an overactive bladder typically involves frequent intravesical instillations, a treatment that patients often find inconvenient or otherwise undesirable. In short, frequent intravesical instillations to restore bladder myocyte function are undesirable, and systemic medications still lack tolerable specificity. Nevertheless, the critical role in bladder function played by the detrusor smooth muscle cells, and their accessibility across the urothelium through intravesical instillations, make them excellent targets for molecular intervention in the treatment of bladder dysfunction.

Because erectile dysfunction and bladder dysfunction are largely caused by altered smooth muscle tone, a method of gene transfer which targets the genes involved in the regulation of smooth muscle tone is extremely desirable, for it would provide a new means of alleviating bladder dysfunction and erectile dysfunction. Similarly, a method of gene transfer that targets the genes involved in the regulation of smooth muscle tone would be extremely useful as a means of alleviating other smooth muscle dysfunctions, including, but not limited to, asthma; BPH; coronary artery disease (infused during angiography); genitourinary dysfunctions of the endopelvic fascia, prostate gland, ureter, urethra, urinary tract, and vas deferens; irritable bowel syndrome; migraine headaches; premature labor; Raynaud's syndrome; varicose veins; and thromboangitis obliterans.

Alterations in ion-channel activity are suspected in the etiology of human smooth-muscle-related disorders as diverse as asthma, bladder dysfunction, erectile dysfunction, and hypertension. In all of these tissues, myocyte potassium (K⁺) channels play a central role in mediating the effects on smooth muscle tone of diverse endogenous substances. Genes for more than thirty K⁺ channels, many of which are expressed in smooth muscle, have been identified (Lawson, K., Clinical Science, 91:651-63, 1996; Lawson, K., Pharmacol. Ther., 70(1):39-63, 1996; and Ashcroft, F. M., ed., Ion Channels and Disease: Channelopathies, New York: Academic Press, 2000). At least four K⁺ channel subtypes have been identified in human corporal (penile) smooth muscle. These include (1) the metabolically-gated K⁺ channel (i.e., K_{ATP}), (2) the large-conductance, calcium-sensitive K⁺ channel (i.e., the K_{Ca} or maxi-K channel), (3) a delayed rectifier channel, and (4) a voltage dependent, fast transient “A” current channel (Christ et al. Int. J. Impotence Res. 5: 77-96, 1993; J. Androl. 14: 319-28, 1993).

Christ et al. (U.S. Pat. No. 6,271,211 B1) teach a method for treating penile flaccidity caused by heightened contractility of penile smooth muscle, which comprises introducing directly into a subject's penile smooth muscle cells a DNA sequence encoding the K_{ATP} channel subunit protein Klr-6.2. Similarly, Geliebter et al. (U.S. Pat. No. 6,150,338) teach a method for inducing penile erection, which comprises introducing DNA encoding a maxi-K channel protein into a subject's penile smooth muscle cells. Christ et al. (U.S. Pat. No. 6,239,117 B1) teach a method of treating bladder dysfunction caused by heightened contractility of bladder smooth muscle, which comprises introducing DNA encoding maxi-K channels protein into a subject's bladder smooth muscle cells. However, none of U.S. Pat. Nos. 6,150,338, 6,239,117, and 6,271,211 teach the regulation of smooth muscle tone by use of a voltage-dependent potassium channel protein; a non-large conductance, calcium-sensitive potassium channel protein; or the smooth muscle specific promoter, smooth muscle alpha actin (SMAA), operably linked to DNA encoding a potassium channel protein.

### SUMMARY OF THE INVENTION

The invention provides a method of regulating smooth muscle tone in a subject, comprising the introduction and expression of a DNA sequence comprising a smooth muscle specific promoter, smooth muscle alpha actin (SMAA), operably linked to a sequence encoding a potassium channel protein that regulates smooth muscle tone, in a sufficient number of smooth muscle cells of the subject to regulate smooth muscle tone in the subject.

The invention also provides a method of regulating smooth muscle tone in a subject, comprising the introduction and expression of a DNA sequence encoding a voltage-dependent potassium channel protein that regulates smooth muscle tone, in a sufficient number of smooth muscle cells of the subject to regulate smooth muscle tone in the subject.

The invention further provides a method of regulating smooth muscle tone in a subject, comprising the introduction and expression of a DNA sequence encoding a non-large conductance, calcium-sensitive potassium channel protein that regulates smooth muscle tone, in a sufficient number of smooth muscle cells of the subject to regulate smooth muscle tone in the subject.

Additional objects of the invention will be apparent from the description that follows.

### BRIEF DESCRIPTION OF THE FIGURES

FIG. 1. Therapeutic efficacy of multiple potassium channel subtypes. The ratio of intracavernous pressure (ICP) to blood pressure (BP) at different intensities of cavernous nerve stimulation is shown in retired breeder rats into which nucleic acid encoding either the potassium channel protein Kv1.5 or SK3 was introduced into corporal smooth muscle cells. Results are also shown from age-matched controls (AMC) which did not receive potassium channel protein gene transfer. An ICP/BP ratio greater than 0.6 (dotted horizontal line) commensurate with penile erection was obtained in experimental animals transfected with SK3 or Kv1.5, but not in control animals. mA=milliamperes.

FIG. 2. Efficacy of smooth muscle specific promoter. The ratio of intracavernous pressure (ICP) to blood pressure (BP) is shown at different intensities of cavernous nerve stimulation in retired breeder rats into which nucleic acid (hSlo) encoding the potassium channel protein maxi-K was introduced into corporal smooth muscle cells, in combination with either a general viral (cytomegalovirus) promoter (pVAC/hSlo) or with a smooth muscle specific promoter (smooth muscle alpha actin, SMAA/hSlo). Results are also shown from age-matched controls (AMC) injected with phosphate buffered saline (PBS) with 20% sucrose. An ICP/BP ratio greater than 0.6 (dotted horizontal line) commensurate with penile erection was obtained in both groups of experimental animals, but not in controls. mA=milliamperes.

### DETAILED DESCRIPTION OF THE INVENTION

The present invention provides a method of regulating smooth muscle tone in a subject, comprising the introduction and expression of a DNA sequence comprising a smooth muscle specific promoter, smooth muscle alpha actin (SMAA), operably linked to a sequence encoding a potassium channel protein that regulates smooth muscle tone, in a sufficient number of smooth muscle cells of the subject to regulate smooth muscle tone in the subject, Preferred potassium channel proteins are the large conductance, calcium-sensitive potassium channel protein maxi-K, the metabolically-gated and inward rectifier potassium channel protein K_{ATP}, the voltage-dependent potassium channel protein Kv1.5, and the small conductance, calcium-sensitive potassium channel protein SK3. In preferred embodiments, the smooth muscle cells are corporal smooth muscle cells or bladder smooth muscle cells, and the potassium channel protein is maxi-K. Preferably, using the smooth muscle specific promoter SMAA operably linked to the DNA sequence encoding the potassium channel protein is at least as effective in regulating smooth muscle tone in a subject as using a viral promoter operably linked to the DNA sequence encoding the potassium channel protein.

The invention also provides a method of regulating smooth muscle tone in a subject, comprising the introduction and expression of a DNA sequence encoding a voltage-dependent potassium channel protein that regulates smooth muscle tone, in a sufficient number of smooth muscle cells of the subject to regulate smooth muscle tone in the subject. Voltage-dependent potassium channel proteins include Kv1.1, Kv1.3, Kv1.5, Kv2.1, Kv3.1b, delayed rectifier channel, and a fast transient “A” current channel. In a preferred embodiment, the voltage-dependent potassium channel protein is Kv1.5. Preferably, the DNA sequence further comprises a promoter operably linked to the sequence encoding the voltage-dependent potassium channel protein. Preferably, the promoter is a smooth muscle specific promoter. More preferably, the smooth muscle specific promoter is smooth muscle alpha actin (SMAA).

The invention further provides a method of regulating smooth muscle tone in a subject, comprising the introduction and expression of a DNA sequence encoding a non-large conductance, calcium-sensitive potassium channel protein that regulates smooth muscle tone, in a sufficient number of smooth muscle cells of the subject to regulate smooth muscle tone in the subject. As used herein, a “non-large conductance, calcium-sensitive potassium channel” means an intermediate conductance, calcium-sensitive potassium channel or a small conductance, calcium-sensitive potassium channel. Small conductance, calcium-sensitive potassium channels include SK1, SK2 and SK3. Preferably, the small conductance calcium-sensitive potassium channel is SK3. Preferably, the DNA sequence further comprises a promoter operably linked to the sequence encoding the potassium channel protein. Preferably, the promoter is a smooth muscle specific promoter. More preferably, the smooth muscle specific promoter is smooth muscle alpha actin (SMAA).

As used herein, “regulation” is the modulation of relaxation or the modulation of contraction.

Examples of smooth muscle cells for which the method of gene transfer may be used include, but are not limited to, visceral smooth muscle cells of the bladder, gastrointestinal tract, bronchi of the lungs, penis (corpus cavernosum), prostate gland, ureter, urethra (corpus spongiosum), urinary tract, and vas deferens; as well as the smooth and/or skeletal muscle cells of the endopelvic fascia. The claimed methods of gene transfer may be used in bladder smooth muscle cells, corporal smooth muscle cells, gastrointestinal smooth muscle cells, prostatic smooth muscle, and urethral smooth muscle. Given the many gross histological and physiological similarities in the factors that regulate the tone of smooth muscle tissue and of other vascular tissue, it follows naturally that similar principles would permit the application of the method of gene transfer to the arterial smooth muscle cells of the bladder, gastrointestinal tract, bronchi of the lungs, penis (corpus cavernosum), prostate gland, ureter, urethra (corpus spongiosum), urinary tract, and vas deferens. The present methods of gene transfer may also be applied to venous smooth muscle cells.

The potassium channel protein that is introduced and expressed in the smooth muscle cells does not necessarily have to be a potassium channel protein that is normally expressed in the smooth muscle cells.

The present invention specifically provides a method of gene transfer wherein the potassium channel protein involved in the regulation of smooth muscle tone modulates relaxation of smooth muscle. These potassium channel proteins will enhance relaxation of smooth muscle, and will also decrease smooth muscle tone. In particular, where relaxation is enhanced in penile smooth muscle, an erection will be more easily attained. Similarly, where spontaneous smooth muscle tone is decreased in the bladder, bladder hyperactivity will be decreased. In this embodiment of the invention, the gene transfer method is particularly useful for treating individuals with an overactive bladder, without affecting the ability of the bladder to empty. As used herein, an “overactive bladder” is one that contracts spontaneously so that an individual is unable to control the passage of urine. This urinary disorder is more commonly called urge incontinence, and may include urge incontinence combined with stress incontinence.

In a preferred embodiment of the present methods, the subject has heightened contractility of a smooth muscle and regulation of the tone of the smooth muscle via gene transfer results in less heightened contractility of the smooth muscle in the subject. Preferably, the smooth muscle cells are penile smooth muscle cells or bladder smooth muscle cells.

The present invention specifically provides methods of regulating penile smooth muscle tone in a subject, comprising the introduction, into penile smooth muscle cells of the subject, of a DNA sequence encoding a potassium channel protein involved in the regulation of smooth muscle tone, and expression in a sufficient number of penile smooth muscle cells of the subject to induce penile erection in the subject. In this embodiment, the method of the present invention is used to alleviate erectile dysfunction.

The invention provides a method of treating erectile dysfunction in a subject, comprising the introduction and expression of a DNA sequence comprising a smooth muscle specific promoter, smooth muscle alpha actin (SMAA), operably linked to a sequence encoding a potassium channel protein that regulates corporal smooth muscle tone, in a sufficient number of corporal smooth muscle cells of the subject to regulate corporal smooth muscle tone in the subject and thereby treat the subject's erectile dysfunction. Preferably, the potassium channel protein is maxi-K, K_{ATP}, Kv1.5, or SK3. Most preferably, the potassium channel protein is maxi-K. Preferably, using the smooth muscle specific promoter SMAA operably linked to the DNA sequence encoding the potassium channel protein that regulates corporal smooth muscle tone is at least as effective in treating erectile dysfunction in the subject as using a viral promoter operably linked to the DNA sequence encoding the potassium channel protein.

The invention also provides a method of treating erectile dysfunction in a subject, comprising the introduction and expression of a DNA sequence encoding a voltage-dependent potassium channel protein that regulates corporal smooth muscle tone, in a sufficient number of corporal smooth muscle cells of the subject to regulate corporal smooth muscle tone in the subject and thereby treat the subject's erectile dysfunction. Preferably, the voltage-dependent potassium channel protein is Kv1.5.

The invention further provides a method of treating erectile dysfunction in a subject, comprising the introduction and expression of a DNA sequence encoding a non-large conductance, calcium-sensitive potassium channel protein that regulates corporal smooth muscle tone, in a sufficient number of corporal smooth muscle cells of the subject to regulate corporal smooth muscle tone in the subject and thereby treat the subject's erectile dysfunction. The non-large conductance, calcium-sensitive potassium channel protein can be an intermediate conductance, calcium-sensitive potassium channel protein or a small conductance, calcium-sensitive potassium channel protein. Preferably, the small conductance, calcium-sensitive potassium channel protein is SK3.

Erectile dysfunction may result from a variety of disorders, including neurogenic, arteriogenic, and veno-occlusive dysfunctions, as well as other conditions which cause incomplete relaxation of the smooth muscle.

Furthermore, the present invention specifically provides methods of regulating bladder smooth muscle tone in a subject, comprising the introduction, into bladder smooth muscle cells of the subject, of a DNA sequence encoding a potassium channel protein involved in the regulation of smooth muscle tone, and expression in a sufficient number of bladder smooth muscle cells of the subject to enhance bladder relaxation in the subject. In this embodiment, the method of the present invention is used to alleviate an overactive bladder. An overactive bladder may result from a variety of causes, including neurogenic, myogenic (i.e., alterations in the detrusor myocyte per se that produce increased contractility), or arteriogenic (i.e., vascular insufficiency or ischemia) dysfunctions, as well as other conditions (e.g., diabetic neuropathy, multiple sclerosis, Parkinson's disease, stroke) which promote altered regulation of the smooth muscle of the bladder. A neurogenic bladder dysfunction may manifest itself as partial or complete urinary retention or overflow incontinence. Examples of neurogenic dysfunctions of the bladder include a hypotonic, or flaccid, bladder, and a spastic, or contracted, bladder. These dysfunctions may result from an abnormality, injury, or disease process of the brain, spinal cord (e.g., spina bifida), or local nerve supply to the bladder and its outlet. Disease processes that result in neurogenic bladder dysfunction include benign hyperplasia of the prostate gland (BPH); cerebrovascular accidents; demyelinating or degenerative diseases, such as multiple sclerosis and amyotrophic lateral sclerosis; diabetes mellitus; a ruptured intervertebral disk; syphilis; and brain or spinal cord tumors.

Further provided by the present invention is a method of gene transfer wherein the potassium channel protein involved in the regulation of smooth muscle tone modulates contraction of smooth muscle.

In addition, the present invention provides methods of reducing the effects of inflammation and irritation on the smooth muscle in a subject, comprising the introduction, into the smooth muscle cells of the subject, of a DNA sequence encoding a potassium channel protein involved in the regulation of smooth muscle tone, and expression in a sufficient number of smooth muscle cells of the subject to reduce the effects of inflammation and irritation. For example, the methods provided by the present invention may be used to reduce the symptoms of cystitis of the bladder, such as interstitial cystitis or radiation-induced cystitis of the bladder. Interstitial cystitis is a condition of the bladder that has clinical manifestations of inflammation and irritation. The interstitial cystitis may be caused, for example, by an allergic reaction, an autoimmune disease, or a collagen disease. Furthermore, the methods of gene transfer provided herein may be used, for example, to reduce the effects of inflammation and irritation on the smooth muscle cells of the ureter, urethra, or urinary tract of a subject, which may be caused by a bacterial, fungal, or parasitic infection.

In other embodiments of the invention, the methods of gene transfer described herein may be used to treat other dysfunctions relating to the performance of smooth muscle, including, but not limited to, asthma; coronary artery disease (infused during angiography); genitourinary dysfunctions of the ureter, urethra, urinary tract, and vas deferens; gastrointestinal motility disorders including constipation, diarrhea, or irritable bowel syndrome; migraine headaches; premature labor; Raynaud's syndrome; varicose veins; and thromboangitis obliterans. When used to treat asthma, the present methods of gene transfer may be administered to a subject by way of aerosol delivery using any method known in the art.

In the methods of the present invention, the subject may be an animal or a human, and is preferably human. Preferably, the dysfunction from which the subject suffers is treated by the methods of the present invention.

The DNA sequence of interest may be introduced into a smooth muscle cell by a number of procedures known to one skilled in the art, such as electroporation, DEAE Dextran, monocationic liposome fusion, polycationic liposome fusion, protoplast fusion, DNA-coated microprojectile bombardment, creation of an in vivo electrical field, injection with recombinant replication-defective viruses, homologous recombination, nebulization, using an EYFP vector, and naked DNA transfer by, for example, intravesical instillation. The DNA sequence may be introduced by means of direct injection into a smooth muscle wall. A preferred smooth muscle wall is the wall of the bladder. In addition, smooth muscle cells can be transfected with the DNA sequence ex vivo and the transfected cells can be transplanted into the subject. The cells to be transfected ex vivo can come from the same subject into which the transfected cells are transplanted. It is to be appreciated by one skilled in the art that any of the above methods of DNA transfer may be combined. The DNA sequence can be genomic DNA or cDNA.

In a preferred embodiment of the invention, the DNA is transferred into the smooth muscle cells by naked DNA transfer, using a mammalian vector. “Naked DNA” is herein defined as DNA contained in a non-viral vector. The DNA sequence may be combined with a sterile aqueous solution, which is preferably isotonic with the blood of the recipient. Such a solution may be prepared by suspending the DNA in water containing physiologically-compatible substances (such as sodium chloride, glycine, and the like), maintaining a buffered pH compatible with physiological conditions, and rendering the solution sterile. In a preferred embodiment of the invention, the DNA is combined with a 20-25% sucrose-in-saline solution, in preparation for introduction into a smooth muscle cell.

Where the DNA is transferred into smooth muscle cells of the bladder, it can be introduced into the bladder by intravesical instillation per the urethra, which is a well-established therapy for the treatment of bladder tumors. The DNA solution is then voluntarily withheld by the patient, within the bladder, for a prescribed duration of time. In another embodiment, the DNA is introduced into the endopelvic fascia, prostate, ureter, urethra, upper urinary tract, or vas deferens by instillation or injection transfer, and the ureter, urethra, or upper urinary tract is obstructed so that the DNA solution remains in contact with the internal epithelial layer for a prescribed, period of time. The DNA sequence for expression may also be incorporated into cationic liposomes and directly injected into the smooth muscle cells of the subject.

The present methods may use viral and/or non-viral recombinant vectors. A viral-based vector comprises: (1) nucleic acid of, or corresponding to at least a portion of, the genome of a virus, which portion is capable of directing the expression of a DNA sequence; and (2) a DNA sequence encoding a potassium channel protein involved in the regulation of smooth muscle tone, operably linked to the viral nucleic acid and capable of being expressed as a functional gene product in the target cell. The recombinant viral vectors of the present invention may be derived from a variety of viral nucleic acids known to one skilled in the art, e.g., the genomes of adenovirus, adeno-associated virus, herpes simplex virus (HSV), lentivirus, Semiliki Forest virus, vaccinia virus, and other viruses, including RNA and DNA viruses.

The recombinant vectors of the present invention may also contain a nucleotide sequence encoding suitable regulatory elements, so as to effect expression of the vector construct in a suitable host cell. As used herein, “expression” refers to the ability of the vector to transcribe the inserted DNA sequence into mRNA so that synthesis of the protein encoded by the inserted nucleic acid can occur. Those skilled in the art will appreciate the following: (1) that a variety of enhancers and promoters are suitable for use in the constructs of the invention; and (2) that the constructs will contain the necessary start, termination, and control sequences for proper transcription and processing of the DNA sequence encoding a potassium channel protein involved in the regulation of smooth muscle tone, upon introduction of the recombinant vector construct into a host cell.

The non-viral vectors provided by the present invention, for the expression in a smooth muscle cell of the DNA sequence encoding a potassium channel protein involved in the regulation of smooth muscle tone, may comprise all or a portion of any of the following vectors known to one skilled in the art: pCMVβ (Invitrogen), pcDNA3 (Invitrogen), pET-3d (Novagen), pProEx-1 (Life Technologies), pFastBac 1 (Life Technologies), pSFV (Life Technologies), pcDNA2 (Invitrogen), pSL301 (Invitrogen), pSE280 (Invitrogen), pSE380 (Invitrogen), pSE420 (Invitrogen), pTrcHis A,B,C (Invitrogen), pRSET A,B,C (Invitrogen), pYES2 (Invitrogen), pAC360 (Invitrogen), pVL1392 and pV11392 (Invitrogen), pCDM8 (invitrogen), pcDNA I (Invitrogen), pcDNA I(amp) (Invitrogen), pZeoSV (Invitrogen), pRc/CMV (Invitrogen), pRc/RSV (invitrogen), pREP4 (Invitrogen), pREP7 (invitrogen), pREP8 (Invitrogen), pREP9 (Invitrogen), pREP10 (Invitrogen), pCEP4 (Invitrogen), pEBVHis (Invitrogen), λPop6 EYFP (Clontech), and pBF. Other vectors would be apparent to one skilled in the art.

Promoters suitable for the present invention include, but are not limited to, constitutive promoters, tissue-specific promoters, and inducible promoters. In one embodiment of the invention, expression of the DNA sequence encoding a potassium channel protein involved in the regulation of smooth muscle tone is controlled and affected by the particular vector into which the DNA sequence has been introduced. Some eukaryotic vectors have been engineered so that they are capable of expressing inserted nucleic acids to high levels within the host cell. Such vectors utilize one of a number of powerful promoters to direct the high level of expression. Eukaryotic vectors use promoter-enhancer sequences of viral genes, especially those of tumor viruses. This particular embodiment of the invention provides for regulation of expression of the DNA sequence encoding the protein, through the use of inducible promoters. Non-limiting examples of inducible promoters include metallothionine promoters and mouse mammary tumor virus promoters. Depending on the vector, expression of the DNA sequence in the smooth muscle cell would be induced by the addition of a specific compound at a certain point in the growth cycle of the cell. Other examples of promoters and enhancers effective for use in the recombinant vectors of the present invention include, but are not limited to, CMV (cytomegalovirus), SV40 (simian virus 40), HSV (herpes simplex virus), EBV (Epstein-Barr virus), retrovirus, adenoviral promoters and enhancers, and preferably smooth-muscle-specific promoters and enhancers. One example of a smooth muscle specific promoter is SM22α. A preferred smooth-muscle-specific promoter is smooth muscle alpha actin (SMAA).

The present invention further provides a smooth muscle cell which expresses an exogenous DNA sequence encoding a potassium channel protein involved in the regulation of smooth muscle tone. As used herein, “exogenous” means any DNA that is introduced into an organism or cell. The introduction into the smooth muscle cell of a recombinant vector containing the exogenous DNA sequence may be effected by methods known to one skilled in the art, such as electroporation, DEAE Dextran, cationic liposome fusion, protoplast fusion, DNA-coated microprojectile bombardment, injection with recombinant replication-defective viruses, homologous recombination, and naked DNA transfer by, for example, intravesical instillation.

Any of the methods of DNA transfer described herein may be combined. In addition, the methods described herein may be combined with other therapies to increase efficacy of treatment while lowering the dose requirement and reducing side effects. For example, erectile dysfunction may be treated using a method of transfer of DNA encoding a potassium channel protein as disclosed herein combined with oral therapy using for example VIAGRA®.

The present invention is described in examples in the following Experimental Details Section. That section is set forth to aid in the understanding of the invention, and should not be construed to limit in any way the invention as defined in the claims which follow thereafter.

### Experimental Details

### Erectile Dysfunction Gene Transfer Using Different Potassium Channel Subtypes and a Smooth Muscle Specific Promoter

The efficacy of different potassium channel subtypes to restore erectile capacity was demonstrated in retired breeder rats transfected with the voltage-dependent potassium channel protein Kv1.5, the small conductance, calcium-sensitive potassium channel protein SK3, and the large conductance, calcium-sensitive potassium channel protein maxi-K in combination with a smooth muscle specific promoter.

The rat was selected for the gene transfer studies, as the rat penis has been shown to be functionally, histologically and pharmacologically similar to the human penis (Lesson, et al., Investigative Urology, 3(2):144-45, 1965). Among many known models, the rat is excellent for the study of penile erection (Lesson, et al., Investigative Urology 3(2):144-45, 1965; Quinlan, et al., J. Urol., 141(3):656-61, 1989; Chen, et al., J. Urol., 147:1124-28, 1992; Martinez-Pineiro, et al., European Urology, 25:62-70, 1994), as well as neurogenic and diabetic impotence (Rehman, et al., Am J. Physiol., 41:H1960-71, 1997).

The studies described below use changes in intracorporal pressure (ICP) elicited by electrical stimulation of the cavernous nerve (CN) as a measure of erectile capacity. The validity of the CN stimulation model has been demonstrated in studies showing that similar results following transfection of maxi-K were obtained using either electrical CN stimulation or electrical stimulation of the medial preoptic area of the brain (Sato, et al. J. Urol. 163(4): Suppl., p. 198, 2000) or in response to chemical (apomorphine) evoked changes in ICP in awake animals (Sato, et al. J. Urol. 165(5): Suppl., p. 220, 2001),

### 1. General Experimental Procedures

Retired breeder Sprague-Dawley rats were used (weight range 500-7000. All rats were fed Purina lab rodent chow ad libitum, and housed individually with a 07:00-19:00 light cycle.

DNA encoding the potassium channel protein was injected into the corpus cavernosum of anesthetized rats as described below. One week after injection, the rats were again anesthetized and underwent an experimental protocol to investigate whether changes in intracavernous pressure (ICP) could be obtained that are commensurate with penile erection.

Rats were anaesthetized by intraperitoneal injection (35 mg/kg) of sodium pentobarbital (Anpro Pharmaceuticals). Anesthesia was maintained during the course of the experimental protocol (2-3 hrs) by subsequent injection of pentobarbital (5-10 mg/kg) every 45-60 minutes, as required.

Surgical preparation for experimental protocol and placement of pressure-monitoring cannulas: Anaesthetized animals were placed in the supine position. The bladder and prostate were exposed through a midline abdominal incision. An arterial line was inserted in the left carotid artery for continuous monitoring of blood pressure (BP). A right external jugular venous line was utilized for intravenous fluid transfusion or blood sampling. The cavernous nerve was located on the posterolateral surface of the prostate, arising from the pelvic: ganglion that is formed by the joining of the hypogastric and pelvic nerves. A nerve-stimulator probe was placed around the cavernous nerve for current stimulation. The two corpora were exposed by inguinoscrotal incisions on both sides, combined with degloving of the penis. In order to monitor intracorporal pressure (ICP), a 23-gauge cannula was filled with 250 U/ml of heparin solution, connected to PE-50 tubing (Intramedic, Becton Dickinson), and inserted into the right corpus cavernosum.

Both pressure lines, BP and ICP, were connected to a pressure transducer, which was, in turn, connected via a transducer amplifier (ETH 400 CB Sciences, Inc.) to a data acquisition board (MacLab/8e, ADI Instruments, MA). Real-time display and recording of pressure measurements were performed on a Macintosh computer (MacLab software v3.4). The pressure transducers and data acquisition board were calibrated in cm of H₂O.

Neurostimulation of cavernous nerve and recording of intracorporal pressure: Direct electrostimulation of the cavernous nerve was performed with a stainless-steel bipolar hook electrode. Each probe was 0.2 mm in diameter; the two poles were separated by 1 mm. Monophasic rectangular pulses were delivered by a signal generator (custom-made, with built-in constant current amplifier). Stimulation parameters were as follows: frequency, 20 Hz; pulse width, 0.22 msec; duration, 1 min. The current protocol involved the application of increasing current at the following intervals: 0.5, 1, 2, 4 and 6 mA. The changes in intracorporal pressure and systemic blood pressure were recorded at each level of neurostimulation.

Statistical comparisons at each level of nerve stimulation were subjected to a One Way ANOVA, with Fischer's Protected Least Significant Difference test used for Post-hoc pair wise comparisons. All differences were considered significant at p<0.05. Data are expressed as the mean (±S.E.M.).

Stimulus-response curves were generated to illustrate the effects of neurostimulation on intracorporal pressure by expressing the change in intracorporal pressure as a function of the mean systemic blood pressure (expressed as ICP/BP), then plotting this ratio as a function of the magnitude of neurostimulation. All data were plotted using Sigma Plot, software for Macintosh computers (Sigma Plot, Jandel Scientific, San Rafael, Calif.).

### 2. Experiments with Kv1.5 and SK3 Potassium Channel Subtypes

Kv1.5 is a voltage-dependent potassium channel that is a member of the superfamily of voltage-sensitive K channels found in many excitable cells (Hille, In: Ion Channels of Excitable Membranes, Sinauer Associates, Inc, Sunderland, Mass., 2002). Kv1.5 has been shown to be present in rat corporal tissue (Archer, Vascul. Pharmacol. 38:61-71, 2002). The potassium channel SK3 is one isoform of a family of small conductance calcium-sensitive potassium channels found in excitable cells (Hille, id.; Herrera & Nelson, J. Physiol. 541(Pt 2):483-92, 2002), including smooth muscle (Ro et al., Am J Physiol Gastrointest Liver Physiol 281(4):G964-73, 2001). SK3 has not been shown to be present in rat or human corporal tissue.

Material and Methods: DNA encoding rat SK3, cloned into plasmid pBF, was obtained from Dr. John Adelman (Kohler M. et al. Science 273 (5282): 1709-14, 1996). DNA encoding human Kv1.5 was cloned by the inventors. The cloned DNA encoding Kv1.5 was inserted into the pVAX expression vector (Invitrogen), a 3.0-kb plasmid vector. The pVAX1 was constructed by modifying the pcDNA3.1 vector to use kanamycin instead of ampicillin for selection, so as to avoid the potential pitfall of sensitivity to penicillin when injecting in humans. The unnecessary sequences for replication in E. coli, or for expression of the recombinant protein, were also removed. The gene Kv1.5 was isolated by PCR amplification using specific primers. It was first cloned in pCR2.1-TOPO for sequencing, and then subcloned using Hind III×BamIII restriction sites into pVAX (Invitrogen) treated with alkaline phosphatase to reduce the background. 100 μg of pVAX/Kv1.5 (n=5) or pBF/SK3 (n=6) were injected into the corpus cavernosum of anesthetized rats. One week after injection, cavernosometry was performed on all rats. The responses obtained in treated rats were compared to responses obtained with age-matched control (AMC) rats injected with vehicle only (i.e., phosphate buffered saline with 20% sucrose).

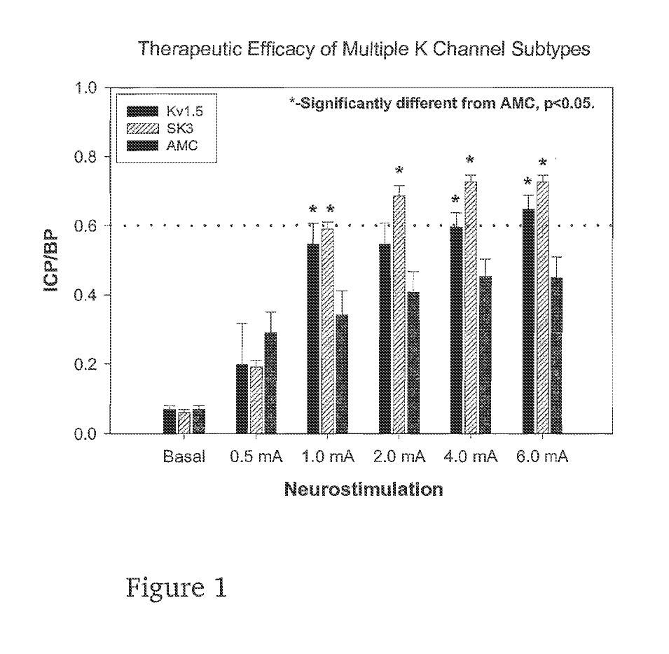

Results: The results of the experiments are shown in FIG. 1. As illustrated in the Figure, the cavernous nerve stimulated intracavernous pressure (ICP) responses were significantly greater in gene transfer experiments with both Kv1.5 or SK3 channel subtypes than in age-matched controls (AMC) at most levels of nerve stimulation (i.e., ≧1.0 mA). Gene transfer with both Kv1.5 or SK3 channel subtypes produces a ratio of intracavernous pressure (ICP) to blood pressure (BP) commensurate with sufficient relaxation of penile smooth muscle to ensure a penile erection adequate for coitus (intercourse). An ICP/BP ratio>0.6 (dashed horizontal line in FIG. 1) is sufficient to ensure erection (Melman et al., J. Urol. 170(1): 285-90, July, 2003).

### 3. Experiments with Maxi-K Potassium Channel in Combination with a Smooth Muscle Specific Promoter

Material and Methods: Experiments were conducted to compare the efficacy of the potassium channel protein maxi-K in restoring erectile capacity when maxi-K was coupled with either a high-efficiency viral promoter or a smooth muscle specific promoter. The smooth muscle specific promoter selected for use in these experiments was smooth muscle alpha actin (SMAA) (Cogan et al., J. Biol. Chem. 270:11310-21, 1995). The general viral promoter used was cytomegalovirus (CMV). The pCMVβ and pcDNA3 plasmids were purchased from invitrogen (San Diego, Calif.). The human cDNA of hSlo, the α- or pore-forming subunit of maxi-K, was obtained from Dr. Salkoff (Washington University School of Medicine, St. Louis, Mo.) (McCobb, et al., American Journal of Physiology, 269: H767-H777, 1995). The nucleotide sequence of the hSlo cDNA is also available at Genbank Accession No. U23767. The human maxi-K channel cDNA (approximately 3,900 nucleotides, or 3.9 kb, long) (McCobb, et al., American Journal of Physiology, 269: H767-H777, 1995) was inserted into the XhoI-XbaI cloning sites of the pcDNA3 vector, where expression is driven off the cytomegalovirus CMVβ promoter (Invitrogen). In the vector SMAA/EYFP, (from John Szucsik, Medical Center of Cincinnati, USA), the EYFP gene was removed and hSlo was inserted in its place to give the plasmid SMAA/hslo. pSMAA/EYFP itself was derived from pSMP8 (described in Cogan et al., J. Biol. Chem. 270: 11310-21, 1999) by inserting the SMAA promoter sequence into an EYFP vector commercially available from Clontech. The plasmid, SMAA/hslo expresses hslo from the SMAA promoter and has a Kanamycin-resistance gene identical to that found in pVAX. 100 μg of pVAX/hSlo (n=7) or SMAA/hSlo (n=5) were infected into the corpus cavernosum of anesthetized rats. Experiments were also conducted in vitro with different cell types to demonstrate the specificity of SMAA/hSlo.

Results: SMAA/hSlo specifically expressed in cultured human corporal smooth muscle cells in vitro, but not in a non-smooth muscle cell type, i.e., human embryonic kidney (HEK) cells, a commonly used cell expression system. These data document the selectivity of SMAA/hSlo expression in human smooth muscle cells.

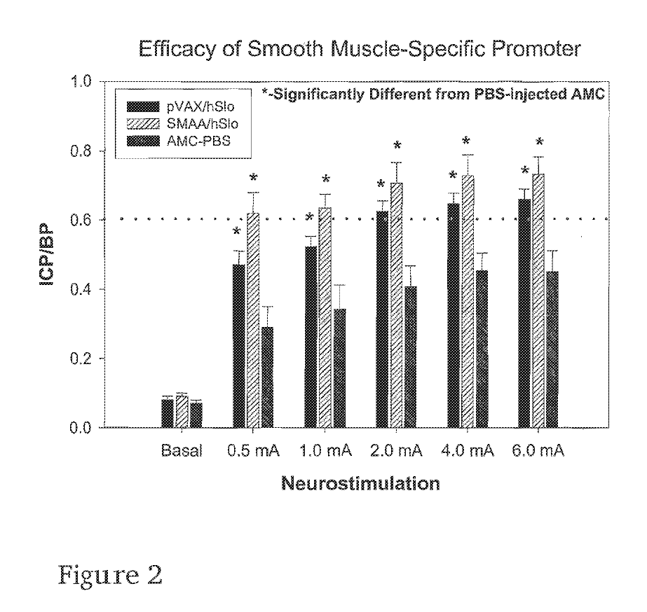

As shown in FIG. 2, gene transfer in vivo into the corpus cavernosum with both types of promoters produces an ICP/BP ratio commensurate with an erection; that is, an ICP/BP ratio >0.6. Thus, in both cases the changes are not only statistically significant, they are physiologically relevant. Use of the smooth muscle specific vector can produce an effect that is equivalent to using a high-efficiency viral promoter.

### 4. General Discussion

The method of gene transfer provided by the present invention is designed to take advantage of the fact that relatively subtle alterations in the balance between contracting and relaxing stimuli can result in profound alterations in smooth muscle tone and function (Christ, et al., British Journal of Pharmacology, 101(2):375-81, 1990; Azadzoi, et al., J. Urol., 148(5):1587-91, 1992; Lerner, et al., J. Urol., 149(5.2):1246-55, 1993; Taub, et al., J. Urol., 42:698, 1993; and Christ, G. J., Urological Clinics of North America, 22(4):727-45, 1995). The goal of gene transfer is to restore a more normal balance between contracting and relaxing stimuli following expression of an exogenous gene that codes for physiologically-relevant potassium channel proteins in smooth muscle. In light of the multifactorial nature of erectile and bladder dysfunctions in humans, there may, in fact, be more than one distinct genetic therapy strategy that will be effective in the restoration of erectile potency or bladder function. Expression of transfected potassium channel protein has been sustained as tested for as long a period as 6 months (Melman et al., J. Urol. 170(1): 285-90, July, 2003). Thus, a patient could obtain “normal” erections, or “normal” bladder function, in the absence of any other exogenous manipulation, during this time period. Clearly, this would be a major advance over all other currently-available therapies. Indeed, the accessibility of the urogenital organs, and the fact that subtle alterations in the tone of smooth muscle are responsible for many aspects of human urogenital disease, all provide clear advantages to the use of gene transfer for treating urogenital disorders.

Studies have now shown that potassium channel subtypes from the major potassium channel families are effective in enhancing relaxation of penile smooth muscle. Specifically, the potassium channels that were tested are the small conductance, calcium-sensitive SK3 (FIG. 1), the large conductance, calcium-sensitive maxi-K (U.S. Pat. No. 6,150,338), the voltage-dependent Kv1.5 (FIG. 1), and the inward rectifier K_{ATP} (U.S. Pat. No. 6,271,211 B1) potassium channels. Taken together, the foregoing data are consistent with the supposition that increased potassium channel activity, following a single intracorporal injection of DNA encoding the potassium channel protein, results from the presence of a greater number of K⁺ channels in some fraction of corporal smooth muscle cells. In turn, this results in a greater hyperpolarization for any given level of endogenous or exogenous stimulus, presumably altering intracellular calcium mobilization/homeostasis, and thereby promoting greater corporal smooth-muscle relaxation. It seems reasonable to assume that the relatively stable transfection of smooth muscle cells with the human K⁺ channel cDNA represents an important and physiologically-relevant strategy for the molecular manipulation of smooth muscle tone in the treatment of smooth muscle disorders such as, for example, erectile dysfunction and bladder dysfunction.

The present application also demonstrates that gene transfer of a potassium channel protein using a smooth muscle specific promoter is capable of restoring erectile capacity in a manner that appears virtually indistinguishable from that observed using a more general viral promoter. However, the use of a vector containing a smooth muscle specific promoter, as opposed to a non-tissue specific promoter, confers additional safety advantages to a gene transfer approach to the treatment of smooth muscle disorders, which is clearly advantageous for the treatment of human subjects.

All of the publications and references cited hereinabove are hereby incorporated by reference in their entirety into the specification.

1. A method of regulating smooth muscle tone in a subject, comprising the direct introduction of a plasmid comprising a DNA sequence comprising a smooth muscle alpha actin (SMAA) promoter operably linked to a hSlo sequence encoding a maxi-K potassium channel protein and expression of the DNA sequence in a sufficient number of smooth muscle cells of the subject to regulate smooth muscle tone in the subject.

2-3. (canceled)

4. The method of claim 1, wherein the smooth muscle cells are arterial smooth muscle cells, venous smooth muscle cells, or visceral smooth muscle cells.

5. The method of claim 4, wherein the smooth muscle cells are located in the bladder, blood vessel wall, gastrointestinal tract, bronchi of the lung, endopelvic fascia, penis, prostate gland, ureter, urethra, uterus, or vas deferens of the subject.

6. The method of claim 4, wherein the visceral smooth muscle cells are bladder smooth muscle cells, corporal smooth muscle cells, gastrointestinal smooth muscle cells, prostatic smooth muscle cells, or urethral smooth muscle cells.

7. The method of claim 1, wherein the DNA sequence is genomic DNA or cDNA.

8. The method of claim 1, wherein the potassium channel protein modulates relaxation of the smooth muscle.

9. The method of claim 8, wherein the potassium channel protein modulates relaxation of corporal smooth muscle.

10-19. (canceled)

20. The method of claim 1, wherein the DNA sequence is introduced by naked DNA transfer.

21. (canceled)

22. The method of claim 1, wherein the DNA sequence is introduced by means of direct injection into a smooth muscle wall.

23. The method of claim 22, wherein the smooth muscle is the bladder.

24. (canceled)

25. The method of claim 1, wherein the subject has heightened contractility of a smooth muscle and regulation of the tone of the smooth muscle results in less heightened contractility of the smooth muscle in the subject.

26. The method of claim 25, wherein the smooth muscle cells are penile smooth muscle cells or bladder smooth muscle cells.

27. The method of claim 1, wherein the subject has an erectile dysfunction or a bladder dysfunction.

28-29. (canceled)

30. The method of claim 27, wherein the erectile dysfunction results from incomplete relaxation of smooth muscle due to neurogenic dysfunction, arteriogenic dysfunction, and/or veno-occlusive dysfunction.

31. (canceled)

32. The method of claim 27, wherein the bladder dysfunction results from bladder overactivity.

33. The method of claim 27, wherein the dysfunction is treated.

34. (canceled)

35. A method of treating erectile dysfunction in a subject, comprising the direct introduction of a plasmid comprising a DNA sequence comprising a smooth muscle alpha actin (SMAA) promoter operably linked to a hSlo sequence encoding a maxi-K potassium channel protein and expression of the DNA sequence in a sufficient number of corporal smooth muscle cells of the subject to enhance relaxation of corporal smooth muscle in the subject and thereby treat the subject's erectile dysfunction.

36-42. (canceled)

43. The method of claim 1, wherein using the smooth muscle alpha actin (SMAA) promoter operably linked to a DNA sequence encoding the potassium channel protein is at least as effective in enhancing relaxation of the smooth muscle in a subject as using a viral promoter operably linked to the DNA sequence encoding the potassium channel protein.

44. The method of claim 35, wherein using the smooth muscle alpha actin (SMAA) promoter operably linked to a DNA sequence encoding the potassium channel protein that enhances relaxation of corporal smooth muscle is at least as effective in treating erectile dysfunction in a subject as using a viral promoter operably linked to the DNA sequence encoding the potassium channel protein.

45. The method of claim 43, wherein the viral promoter is a cytomegalovirus (CMV) promoter.
